(19) United States
(12) Patent Application Publication
(10) Pub. No.: US 2025/0140340 A1
(43) Pub. Date: May 1, 2025

(54) Methods For Identifying Microsatellite Instability High (MSI-H) IN DNA Samples

(71) Applicant: Canexia Health Inc., Vancouver (CA)

(72) Inventor: Dilmi Chathurika PERERA, Vancouver (CA)

(21) Appl. No.: 18/682,487

(22) PCT Filed: Aug. 8, 2022
(86) PCT No.: PCT/IB2022/057382

§ 371 Date: Feb. 9, 2024

Related U.S. Application Data
(60) Provisional application No. 63/231,098, filed on Aug. 9, 2021.

(51) Int. Cl. G16B 20/20; G06N 20/00; G16B 40/20

(52) U.S. Cl. CPC G16B 20/20; G06N 20/00; G16B 40/20

(57) ABSTRACT

A method is proposed where the distribution of repeats relative to the negative control sample in each run are calculated for each MSI loci. This distribution along with the average, are used as features to train a random forest classifier to identify MSI-H samples from other samples which are either MSS or MSI-L samples, collectively referred to as MS-Stable. In particular, the method distinguishes samples subject to artificial replication errors, such as PCR errors and sequencing errors. The resulting output that is produced is the probability of the sample being MSI-H, which is the MSI score of the sample. When samples are classified as MSI-HI, the finding is reported as indicative of sensitivity to immune modulation-checkpoint inhibitor treatment.

### CROSS REFERENCE TO RELATED APPLICATIONS

This application claims the benefit of priority of U.S. Provisional Patent Application No. 63/231,098 filed on Aug. 9, 2021, which is hereby incorporated by reference.

### FIELD

The present disclosure relates generally to bioinformatics. More particularly, the present disclosure relates to a computational method for detecting microsatellite instability high (MSI-H) tumours using next-generation sequencing (NGS) data.

### BACKGROUND

In genomes there are tracts of repetitive DNA in which certain nucleic acid sequence patterns ranging in length from 1 to 6 or more base pairs are repeated. These repetitive DNA tracts are referred to as a microsatellite (MS). Within a microsatellite, the repetition of the sequence pattern can occur anywhere between 5-50 times by example. FIG. 1 is an illustration of a portion of a DNA sequence where the circled region 10 highlights an example MS where the sequence of “CT” repeats 11 times. In the genome, there can be numerous microsatellite occurrences, where each can be assigned a sequential position number relative to a particular read direction (forward or reverse). For the purposes of this description, each MS position is referred to as an MS locus.

In particular, these regions are susceptible to DNA replication errors which are corrected by the DNA mismatch repair machinery. When the DNA mismatch repair machinery is deficient, such as in DNA mismatch repair deficient tumors (MMRd/MSI-H), these regions will have higher rates of DNA replication errors resulting in alleles with different numbers of repeats, which can be identified using NGS (next-generation sequencing). Hence such samples which have high microsatellite instability (MSI-H) due to the presence of these DNA replication errors are of interest because they can be indicators of cancerous tumors that are sensitive to certain types of cancer treatments (e.g., immune modulation-checkpoint inhibitor treatment). There are tumors with low levels of microsatellite instability which are sometimes classified as MSI-L. Unlike MSI-H tumors, MSI-L tumors are not indicated as eligible for these treatments and are considered to be phenotypically similar to microsatellite stable (MSS) tumors. Therefore, in this analysis these MSI-L tumors and MSS tumors are grouped into one group, collectively referred to as MS-Stable tumours.

FIGS. 2A and 2B illustrate examples of the DNA sequence shown in FIG. 1 after replication errors have occurred, such as during the PCR process. In the example of FIG. 2A, the improper replication has resulted in a shorter number of repetitions in circled region 12 relative to region 10. In the example of FIG. 2B, the improper replication has resulted in a longer number of repetitions relative to region 10 in circled region 14. These are but 2 examples of improper replication, and the improper replication could result in an even smaller or larger number of repetitions relative to region 10.

Persons of skill in the art will understand that PCR (Polymerase Chain Reaction) is used to amplify samples for the purposes of rapid replication of DNA for molecular and genetic analysis. From the result of PCR, it is desirable to either observe MSI-H samples, or alternately the lack of such DNA replication errors, to thereby categorize the samples as MS-Stable.

Unfortunately, these MS regions are also more susceptible to PCR errors (stutter peaks) and sequencing errors. Both of these types of artificially introduced errors can result in replication errors that appear indistinguishable from the naturally occurring replication error indicative of a cancerous tumor. More specifically, this could result in replicated DNA having different lengths of MS.

Therefore uncertainty is introduced by the PCR and sequencing process as truly MS-Stable samples may inadvertently appear as MSI-H.

It is therefore desirable to accurately identify true MSI-H samples from MS-Stable samples based on an analysis of these MS regions.

### SUMMARY OF THE INVENTION

In a first aspect, the present invention provides a method for classifying a tissue sample of a person as being microsatellite instability high (MSI-H) without using normal tissue from the same person. The method includes training a machine learning classifier algorithm using known MSI-H samples and known MS-Stable samples to learn a baseline distribution of repeats in microsatellite regions of genomes relative to a first negative control sample at multiple corresponding MS loci, where each of the known MSI-H samples and the known MS-Stable samples are scored with a probability of being MSI-H and the first negative control sample is known to be MS-Stable; setting a threshold probability score in response to groupings of probability scores of the known MSI-H samples and the known MS-Stable samples where the probability scores greater than the threshold probability score are classified as MSI-H; determining a distance of distributions of repeats in the tissue sample normalized to a second negative control sample at multiple MS loci, where both the tissue sample and the second negative control sample are part of the same sequencing run and the second negative control sample is known to be MS-Stable; executing the trained machine learning classifier algorithm on the distance distributions for the tissue sample to provide a corresponding probability score; determining the tissue sample as MSI-H if the score is greater than the threshold probability score; and outputting the MSI-H status of the sample as being indicative of sensitive to immune modulation-checkpoint inhibitor treatment if the score is greater than the threshold.

According to an embodiment of this first aspect, training the machine learning classifier algorithm includes preparing training data for learning by the machine learning classifier algorithm by executing a sequencing run on a plurality of the known MSI-H samples, a plurality of the known MS-Stable samples, and the first negative control sample in the same run. In another embodiment of the first aspect, training the machine learning classifier algorithm includes preparing training data for learning by the machine learning classifier algorithm by executing multiple sequencing runs, each run including a plurality of the known MSI-H samples, a plurality of the known MS-Stable samples, and the first negative control sample.

In yet another embodiment of the first aspect, training the machine learning classifier algorithm includes preparing training data for learning by the machine learning classifier algorithm by executing multiple sequencing runs, each run including a plurality of the known MSI-H samples and the first negative control sample, or a plurality of the known MS-Stable samples and the first negative control sample. In this embodiment preparing the training data includes determining distances between distribution of repeats at the multiple MS loci in each of the known MSI-H samples relative to the first negative control sample, and determining distances between distribution of repeats at the same multiple MS loci in each of the known MS-Stable samples relative to the first negative control sample. The distances can be determined using a distance metric, where the distance metric is a stepwise difference distance metric expressed by

$$d = {\sum }_{γϵ ( {R}_{T} ⋃ {R}_{NC} )} \vert {T}_{γ} - {NC}_{γ} \vert$$

where Tγ is the fraction of reads with repeats of length γ in the tumour sample at a given MS locus, and NCγ is the fraction of reads with repeats of length γ in the NC sample at the same MS locus.

In this embodiment, the threshold probability score is determined by obtaining the optimum threshold that maximizes the true positive rate and minimizes the false positive rate and selecting an adjustment value above it where scores greater than the threshold probability score are classified as MSI-H. According to present aspects, the threshold probability score is a first threshold, and a second threshold is determined by the adjustment value lower than the optimum threshold and samples with scores below this second threshold are classified as MS-Stable. The adjustment value can be determined by a spread of training data scores proximate the optimum threshold. Alternately, the adjustment value is a first adjustment value over the optimum threshold and a second adjustment value is determined as below the optimum threshold. According to this aspect, the first threshold and the second threshold are different, the first threshold is greater than the second threshold, and scores falling between the first threshold and the second threshold are classified as possible MSI. Here outputting the MSI-H status includes outputting the MS-Stable status of the person as MS-Stable or outputting the possible evidence of MSI status of the person as requiring orthogonal testing.

According to another aspect of this embodiment, training the machine learning classifier algorithm includes providing a subset of the training data for execution by the machine learning classifier algorithm. After training with the subset of the training data, validating the trained machine learning classifier algorithm by providing a remainder of the training data that is not the subset of the training data and comparing the probability scores against expected classification results.

In another embodiment of the first aspect, the machine learning classifier algorithm is a random forest classifier, where distributions of the repeats for less than 100 MS loci can be used for training the machine learning classifier algorithm. More specifically, distributions of the repeats for 21 MS loci can be used for training the machine learning classifier algorithm.

In a second aspect, the present invention provides a method of training a machine learning classifier algorithm to classify a tissue sample from a person as being at least MSI-H and indicative of being sensitive to immune modulation-checkpoint inhibitor treatment. The method includes executing multiple sequencing runs, each run including a plurality of the known MSI-H samples and a negative control sample, where the negative control sample is known to be MS-Stable, a plurality of the known MS-Stable samples and the negative control sample, or a combination of the known MSI-H samples the known MS-Stable samples and the negative control sample. The method continues by determining distances between distribution of repeats at multiple MS loci in each of the known MSI-H samples relative to the negative control sample; determining distances between distribution of repeats at the same multiple MS loci in each of the known MS-Stable samples relative to the negative control sample; and providing the distances and their average as features for the machine learning classifier algorithm to learn a baseline distribution of the repeats in the multiple MS loci from the known MSI-H samples and the known MS-Stable samples, and scoring each sample with a probability of being MSI-H.

According to an embodiment of this aspect, the machine learning classifier algorithm is a random forest classifier using the distribution of the repeats as features. In this embodiment, distributions of the repeats for less than 100 MS loci are used for training the machine learning classifier algorithm. More specifically, distributions of the repeats for 21 MS loci are used for training the machine learning classifier algorithm.

In a third aspect, the present invention provides a method for classifying a tissue sample of a person as being microsatellite instability high (MSI-H) without using normal tissue from the same person, using a random forest machine learning classifier algorithm trained using known MSI-H samples and known MS-Stable samples to learn a baseline distribution of repeats in microsatellite regions of genomes relative to a first negative control sample known to be MS-Stable at multiple corresponding MS loci. The method includes determining a distance of distributions of repeats in the tissue sample normalized to a second negative control sample at multiple MS loci, where both the tissue sample and the second negative control sample are part of the same sequencing run and the second negative control sample is known to be MS-Stable; executing the trained machine learning classifier algorithm on the distance distributions for the tissue sample to provide a corresponding probability score; comparing the probability score of the tissue sample to a first predetermined threshold probability score and a second predetermined threshold probability score; and outputting the MSI-H status of the person as a finding indicative of sensitivity to immune modulation-checkpoint inhibitor treatment if the probability score is greater than the first predetermined threshold, or outputting the status of the person as a clinically relevant status if the probability score is less than the second predetermined threshold.

According to an embodiment of this third aspect, the machine learning classifier algorithm is trained and executes using distributions of the repeats for a set number of MS loci less than 100. According to another embodiment of this third aspect, the first predetermined threshold is greater than the second predetermined threshold, and outputting further includes outputting a possible evidence of MSI status of the person as requiring orthogonal testing when the probability score of the tissue sample is at or between the first predetermined threshold and the second predetermined threshold.

Other aspects and features of the present invention will become apparent to those ordinarily skilled in the art upon review of the following description of specific embodiments of the invention in conjunction with the accompanying figures

### BRIEF DESCRIPTION OF THE DRAWINGS

Embodiments of the present disclosure will now be described, by way of example only, with reference to the attached Figures.

FIG. 1 is an illustration of a portion of DNA with a microsatellite region;

FIG. 2A is an illustration of the portion of DNA in FIG. 1 after a replication error occurring in the microsatellite region;

FIG. 2B is an illustration of the portion of DNA in FIG. 1 after a different replication error in the microsatellite region;

FIG. 3 is an example DNA sequence with a microsatellite region;

FIG. 4A is a bar graph plotting the count of occurrences of a number of microsatellite sequences for one MS locus in a first test sample;

FIG. 4B is a bar graph plotting the count of occurrences of a number of microsatellite sequences for the same MS locus in a second test sample;

FIG. 5A is a bar graph plotting the count of occurrences of a number of microsatellite sequences for a specific MS locus in an MS-Stable sample;

FIG. 5B is a bar graph plotting the count of occurrences of a number of microsatellite sequences for the same MS locus with instability in an MSI-H sample;

FIG. 5C is a bar graph plotting the count of occurrences of a number of microsatellite sequences for the same MS locus in a negative control (NC) sample;

FIG. 6 is a graph plotting distribution of MS loci for an MS-Stable sample, MSI-H sample and the NC;

FIG. 7 is a graph re-plotting the MS loci distribution of the MS-Stable and MSI-H data relative to NC of FIG. 6;

FIG. 8 is an alternate plot of distance data using principal component analysis;

FIG. 9 is a flow chart outlining steps of the training phase of the MSI detection system, according to a present embodiment;

FIG. 10 is an example graphical representation of a random forest classifier;

FIG. 11 is an example graphical representation of one decision tree in a random forest classifier with example distance to NC thresholds;

FIG. 12 is a graph plotting experimental results of the MSI detection system based on MSI status known data included in the machine learning training set;

FIG. 13 is a flowchart outlining steps of the detection phase of the MSI detection system, according to the present embodiment;

FIG. 14 is a graph plotting experimental results of the MSI detection system based on MSI status known data not included in the machine learning training set;

FIG. 15A is a graph plotting experimental MSI analytical validation results; and,

FIG. 15B is a graph plotting MSI clinical validation results.

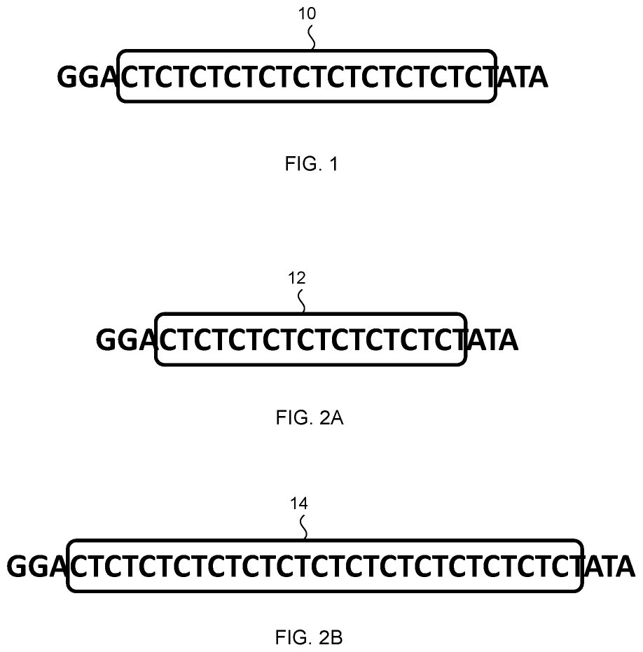

### DETAILED DESCRIPTION

According to the presently described embodiments, in order to identify MSI-H samples from the MS-Stable samples, a machine learning model is trained using the distributions of repeats at MS loci in such a way that the model learns the baseline distribution of repeats, which includes PCR errors and sequencing errors, from MS-Stable samples. The method of the present embodiments uses less than 100 MS loci, which is the minimum number of MS loci that known algorithms use to predict the MSI status of a sample as MSI-H or MS-Stable. Moreover, the method of the present embodiments has been demonstrated with comparable or better accuracy and effectiveness using 21 MS loci.

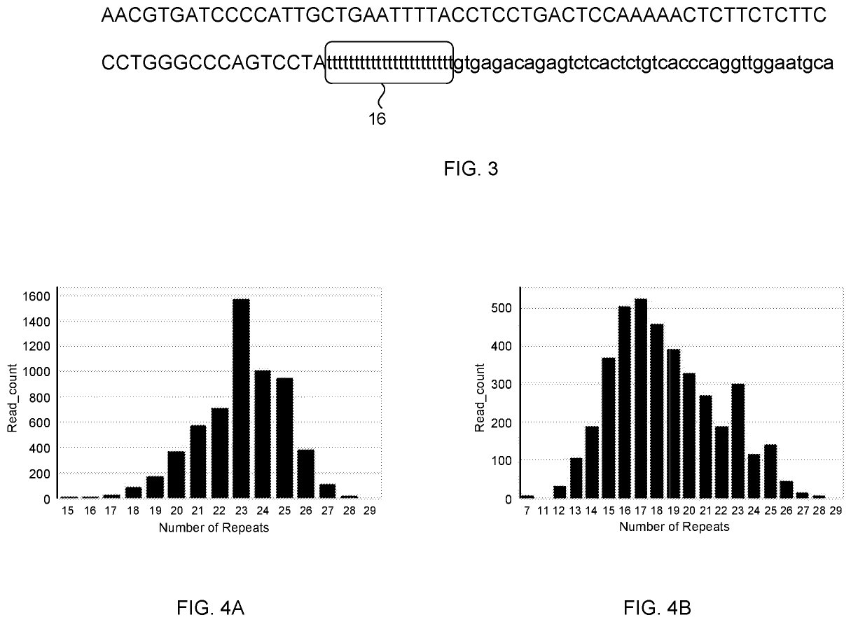

FIG. 3 is an illustration of an example DNA sequence with an MS region 16, shown as a 23 base pair homopolymer. Once this sample is subjected to PCR, the microsatellite region 16 of the amplified DNA can be analyzed by counting the number of times a particular repeating sequence is observed.

FIG. 4A shows one example MS repetition count in a bar chart with the count appearing on the vertical axis, and a specific set of repetition lengths appearing on the horizontal axis which are counted for the MS region 16. In this example, the homopolymer having repetition lengths from 15-29 are plotted. FIG. 4B shows another possible example MS repetition count in a bar chart for the same MS locus, but for a different sample than that of FIG. 4A. It is assumed that both samples have been subjected to PCR in the same sequencing run, meaning that both samples are subjected to the same experimental steps and variances which may occur from run to run.

It can be seen that the distribution of counts across the repetitions for both samples differs. However, it is unclear from looking at both distributions which sample could be MSI-H or MS-Stable.

Commonly used computational methods for analyzing these data for predicting MSI-H or MS-Stable samples includes MANTIS and MSISensor. These are referred to as MSI detection systems or MSI callers, which are computer executable programs/algorithms that implement specific methodologies to distinguish MSI-H from MS-Stable samples. However, these MSI detection systems have proven unsuitable due to high cost and/or poor predictiveness. For example, both the above MSI detection systems each requires a normal tissue/cell sample in addition to the sample under test for determination of MSI-H or MS-Stable status, which doubles the sequencing costs.

It has been noted that some MS regions are more predictive of microsatellite instability than others. For example, repeat sequences greater than 10 bp length are preferred for analysis, as are mononucleotide repeat sequences, etc. However MSI detection systems like MANTIS and MSIsensor use tests such as calculating the fraction of MS regions where the distribution of repeats in the tumour is statistically different from the matched normal (using the χ2 Goodness-of-Fit test) or by calculating the average of the distances between the tumour and the normal at each MS site (using a distance metric such as Euclidean distance, Cosine dissimilarity, stepwise distance, etc.) give equal weights to all MS regions.

In order for simple averaging methods such as those used by MSISensor and MANTIS to be able to provide acceptable levels of predictiveness, a large number of MS loci need to be considered (>100) and filters for selecting more predictive loci (e.g. repeat length>10, use only homopolymer sites, etc.) need to be used, which are not feasible options with a targeted amplicon-based assay.

The number of reads allocated to a particular sample is roughly a fixed amount and having to use more amplicons (e.g. >100 loci) for MSI detection means the number of reads allocated to other amplicons will be reduced. In tumour sequencing applications of targeted amplicon-based assays, greater numbers of reads need to be allocated to other amplicons such as those designed for detecting low prevalent mutations. Therefore, in such application, the ability to detect microsatellite instability with fewer amplicons is beneficial.

Additionally, due to limitations in the primer/amplicon design process for targeted assays, it may not be possible to choose the most predictive MS sites (e.g., if they are in GC (guanine-cytosine) rich regions, if they affect performance of other amplicons in the assay, if they form primer-dimers, etc.). In such applications, being able to use both more predictive and less predictive loci and having a method capable of giving more weight/feature importance to more predictive loci is beneficial.

According to a present embodiment, a novel MSI detection system is provided that does not have the disadvantages of prior MSI detection systems, while providing at least the same accuracy. The presently described embodiments of the MSI detection system includes a training phase and a detection phase. Both phases are configured as computer executable programs or algorithms, and executable on a local network or on a cloud computing platform. The training phase includes a supervised machine learning algorithm that is trained to establish a baseline distribution of repetition counts for the MS loci, resulting in a machine learning model which is then used in the detection phase to differentiate MSI-H samples from MS-Stable samples. Machine learning is considered supervised in this embodiment because data from samples known to be MSI-H and MS-Stable are used for training. Additionally, the machine learning algorithm learns to give higher weights/feature importance to more predictive MS sites, thus eliminating the need to select the most predictive sites or a large number of loci.

Once the algorithm is trained, the resulting machine learning model predicts whether or not the sample under test is MSI-H or MS-Stable. It is noted that the MS-Stable samples can include PCR errors (stutter peaks) and sequencing errors as is the case with MSI-H samples.

It is important to note that the training of the machine learning algorithm and execution of the model only requires a negative control sample and one sample per person, that being a tumor sample, and does not require normal tissue/cells from the same person. Obtaining normal tissue/cells from the same person is costly because of additional storage and sample preparation process involved (e.g. when the tumour sample is a solid tissue sample and the normal sample from the same person is a blood sample), which is in addition to doubling of sequencing costs. Accordingly, an immediate cost advantage is obtained with the present embodiments.

In order to normalize the features used in the machine learning algorithm, the distribution of these repeats is analyzed relative to the distribution of repeats for the negative control sample in a sequencing run for the same corresponding MS locus. The negative control sample is known to be an MS-Stable sample.

The presently described embodiments work under the principle that an MS-Stable sample under test will have distributions at targeted MS loci which are similar to the distributions of the negative control sample. Conversely, an MSI-H sample under test will have distributions that are substantially different from the distributions of the negative control sample at most of the targeted MS loci.

Accordingly, in a present embodiment, the distribution of repeats for the sample under test relative to the negative control sample in each run are calculated for each MS loci, and these features, along with the average, are used to train a random forest classifier to identify MSI-H samples from MS-Stable samples and it produces as output the probability of the sample being MSI-H, which is the MSI score of the sample under test.

This distribution of repeats relative to the negative control sample is referred to in this description as the distance to the NC (Negative Control). The present embodiments are not limited to using a particular negative control sample as long as it is an MS-Stable sample.

Various distance metrics (e.g. Euclidean distance, stepwise difference, etc.) can be used to calculate the distance to NC at a given MS locus for a sample under test. The distance metric used in the present embodiments is the stepwise difference distance metric presented by Kautto E A, Bonneville R, Miya J, Yu L, Krook M A, Reeser J W, et al. Performance evaluation for rapid detection of pan-cancer microsatellite instability with mantis. Oncotarget (2017) 8:7452-63. doi: 10.18632/oncotarget.13918 (Kautto et al, Oncotarget 2017).

Distance to NC at a given MS locus (d) can be expressed as:

$$d = {\sum }_{γϵ ( {R}_{T} ⋃ {R}_{NC} )} \vert {T}_{γ} - {NC}_{γ} \vert$$

where
Tγ—Fraction of reads with repeats of length γ in the tumour sample at a given MS locus
NCγ—Fraction of reads with repeats of length γ in the NC sample at the same MS locus

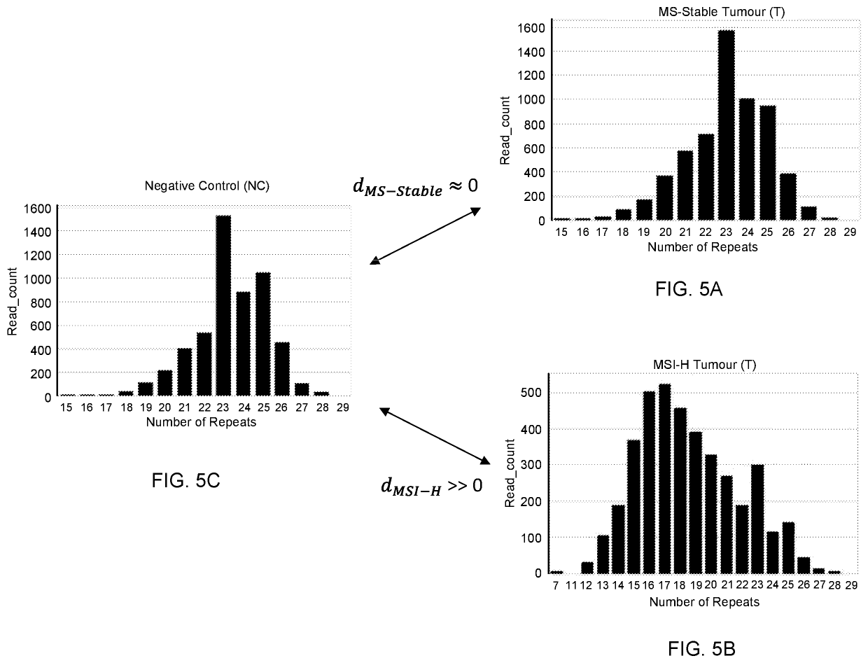

FIGS. 5A, 5B and 5C are bar charts showing the example distribution of repeats at the same specific locus in an MS-Stable sample (FIG. 5A), MSI-H sample (FIG. 5B) and the negative control (FIG. 5C). It is noted that the number of repeats plotted on FIG. 5A and FIG. 5C are from 15-29, while the number of repeats plotted on FIG. 5B includes 7 and 11-29. As seen from the plots, the distribution of repeats in the negative control of FIG. 5C is closer to the MS-Stable sample of FIG. 5A than the MSI-H sample of FIG. 5B (d=distance between distribution of repeats in NC and tumour sample at the given MS locus). More specifically for example, most of the sequenced reads that align to this locus in the MS-Stable sample have 23 repeats (see FIG. 5A), which is the expected number of repeats for this MS locus. In the MSI-H sample of FIG. 5B, the distribution is skewed to the lower end of the distribution with most of the reads having only 16-18 repeats. Therefore, at this MS site, the distance to NC in the MS-Stable sample will be close to 0 (d_{MS-Stable}≈0) and distance to NC in the MSI-H sample will be greater than 0 (d_{MSI-H}>>0).

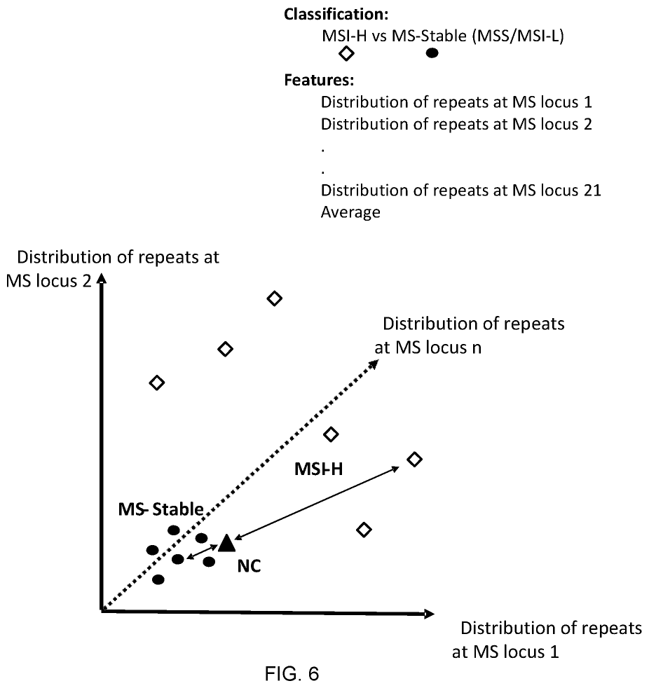

FIG. 6 is a schematic n-dimensional plot representing some measure of the overall distribution of repeats at n MS loci for MS-Stable samples, MSI-H samples and an NC sample. The MSI-H data points are shown as diamonds while the NC data point is shown as a triangle. The remaining circular data points are MS-Stable samples. It can be seen that distance to the NC by all of the MS-Stable samples are generally less than the distance to the NC by all of the MSI-H samples.

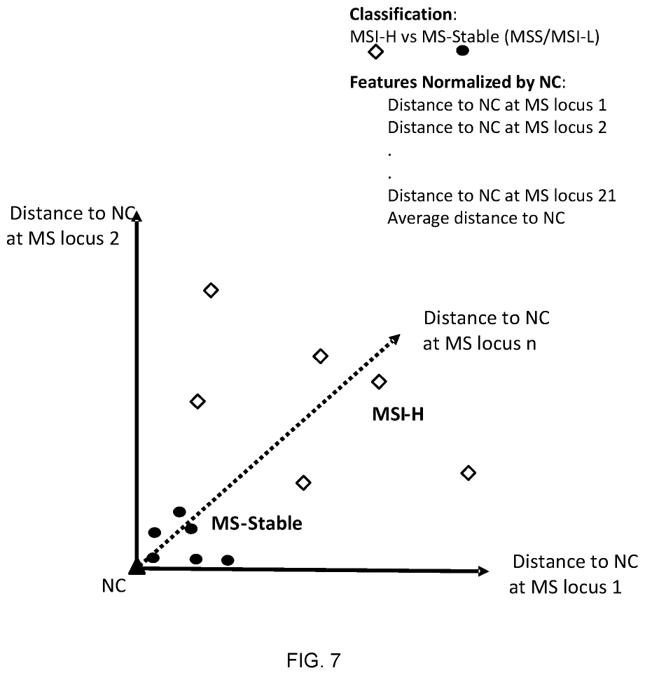

FIG. 7 is a schematic n-dimensional plot of the data from FIG. 6 relative to NC, showing how distance to the NC by all of the MS-Stable samples are generally less than the distance to the NC by all of the MSI-H samples.

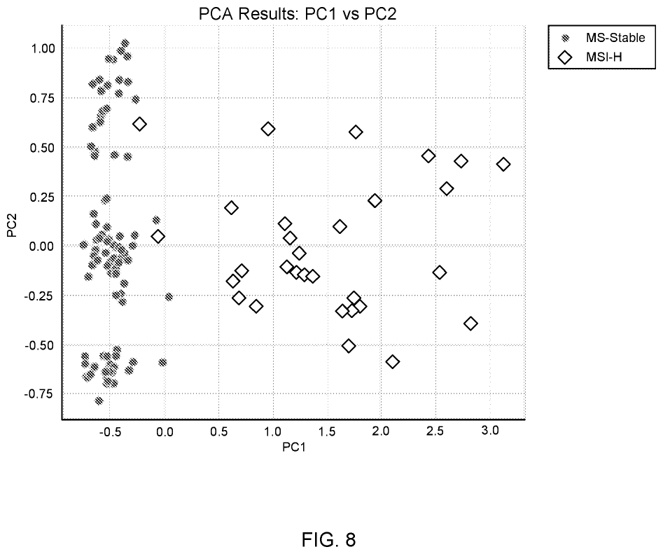

The 22-dimensional feature space represented by the schematic in FIG. 7 can be reduced to a two-dimensional space using principal component analysis (PCA). FIG. 8 is an example plot of distance to NC data for a multitude of samples but using principal component analysis (PCA): PC1 versus PC2. In FIG. 8 all the MSI-H samples are shown as diamonds where most have a PC1 value greater than 0.5. The remaining plotted samples are MS-Stable. It can be seen from FIG. 8 the clustering of the MSS samples closer to each other, while the MSI-H samples are more randomly distributed and further apart from each other. Therefore, it can be concluded that an assumption that the negative control sample, which is also an MS-Stable sample, would be closer to the MS-Stable samples in this feature space holds true. According to the PCA plot, there is also a clear separation of the MS-Stable and MSI-H samples in this feature space, which demonstrates the feasibility of training an ML (machine learning) classifier algorithm to differentiate between the two groups using these 22 features.

A computer system is programmed to calculate distance to NC (d) at each MS locus of interest for the sample under test. These values of d become the data set to be analyzed to determine if the sample under test is MSI-H or MS-Stable. Alternatively, the analysis can provide a further result indicating that the sample under test is neither MSI-H nor MS-Stable, but something in between indicative of possible evidence of microsatellite instability. Examples of two such cases can be seen in the PCA plot in FIG. 8 as the two diamonds plotted between −0.5 and 0.0. These are two MSI-H samples that are located in the intermediate space between the two groups, further away from the other MSI-H samples.

Samples categorized as neither being definitively MSI-H or MS-Stable may have a low signal of MSI, either due to low tumor purity/fraction of the sample or low level of instability in the MS sites. These samples will then be flagged for orthogonal testing (i.e., to be tested with another method such as IHC or MSI PCR which are standard of care for cancer patients) to further determine their relevance.

Application of the distance to NC for each locus of interest is executed by the computing system to classify the sample as noted above. Different machine learning classifiers (i.e., random forest, logistic regression and support vector machines) can be trained and performance can be evaluated by performing k-fold cross validation on the training set described above. Experimental results executed using different classifiers shows that all classifiers exhibited similar performance during cross-validation; however, the random forest classifier showed better separation between the two classes. From this point forward, reference to the random forest classifier is intended to refer to either the algorithm or resulting trained model depending on the context of its discussion.

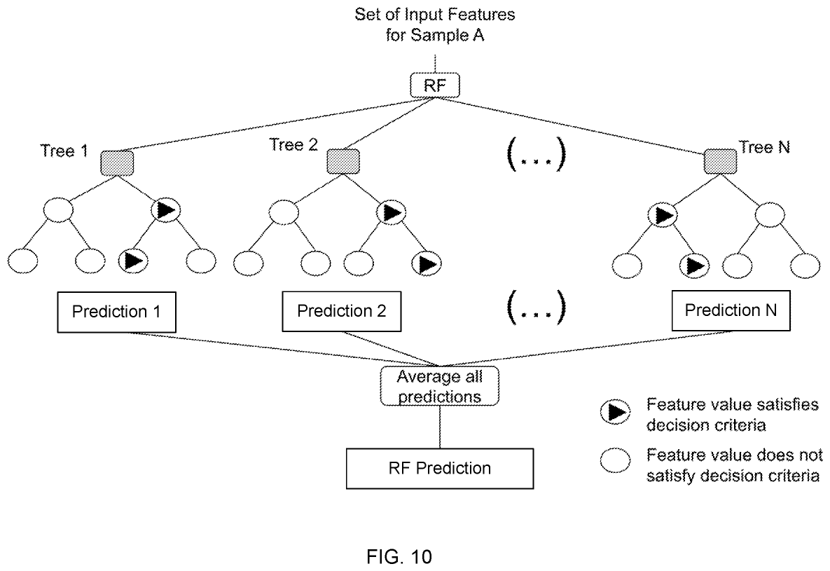

FIG. 10 is a graphical illustration of a generic random forest classifier decision tree. The random forest classifier is a well-known algorithm in the art, and in the present embodiments, is trained using data from known MSI-H samples and known MS-Stable samples (through IHC or MSI PCR) sequenced using the FIND IT™ v5 assay developed by Canexia Health Inc. on the MiSeq™ and NextSeq™ instruments developed by Illumina. In one example, the MS-Stable samples are a mix of buffy coat (normal cells from solid tumor patients) and tumor samples. All of the clinical samples are subjected to PCR along with the negative control sample. The nodes shown with a triangle are feature values that satisfy the decision criteria. The remaining nodes without triangles are feature values that do not satisfy the decision criteria.

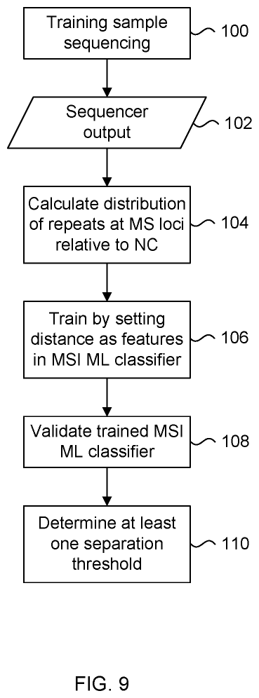

The principles described above are applied to a method for training an MSI ML classifier of the present embodiments. Reference is made to FIG. 9 which is a flow chart outlining the steps of the training phase of the MSI detection system, according to a present embodiment.

The training phase can begin with training sample sequencing step 100, where samples known in advance to be MSI-H and MS-Stable, and a negative control sample are sequenced using any known sequencing instrument. It is assumed that the known MSI-H and MS-Stable samples, and the negative control sample have been subjected to PCR and sequencing in the same run.

The resulting sequenced data is recorded and output as file 102. Alternately, training sample sequencing step 100 can be executed in another lab or location with the resulting file 102 transferred as an input to the next step.

To generate the training data, calculations are made for the distance to NC at each of 21 MS sites by the known MSI-H and MS-Stable samples at step 104. These are the features or the x values of the ML classifier, which in the present embodiments is the random forest classifier. There are 22 x values for a given sample (distance to NC for 21 MS sites+average distance). The prediction class or y value of the random forest classifier is the MSI status (determined beforehand using another test like IHC or MSI PCR).

The features of the algorithm above consists of distance to NC at a particular MS site. Every run has an NC sample and for all the MSI status known samples for a given run in the training set, the distance to NC is calculated using the NC sample in that run. This is a way to normalize the x variables from run-to-run variation.

The random forest classifier is trained on these normalized features at step 106, and it learns the baseline variation from MS-Stable samples and to differentiate that from the variation of MSI-H samples.

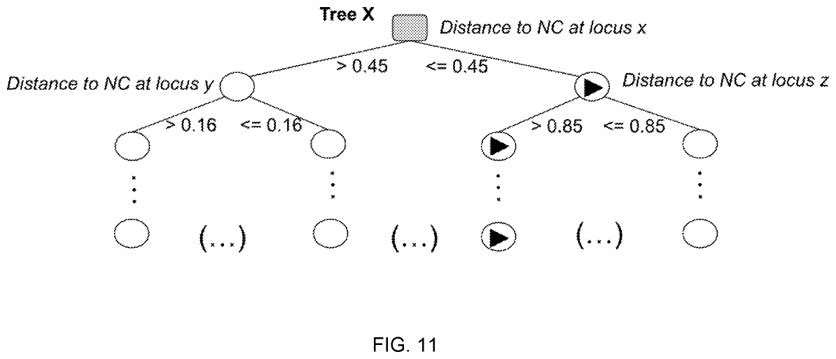

FIG. 11 is an example decision tree of the random forest classifier above after training at step 106. Example distance fractions (d) are annotated, which are arrived at by the learning from the training data. The nodes shown with a triangle are featured values that satisfy the decision criteria. The remaining nodes without triangles are featured values that do not satisfy the decision criteria.

The trained random forest classifier is now validated at step 108. Using 10-fold cross-validation (i.e., The random forest classifier is trained on 90% of the training set and the trained model is validated (predictions are obtained and compared with the expected output) on the remaining 10%. This process is repeated iteratively until all samples are used in both training and validation), 10 random forest classifiers are trained, and a cross-validation ensemble is created where, the final probability score prediction for a given sample is obtained by taking the average of the probability score predictions from the 10 trained models for that sample.

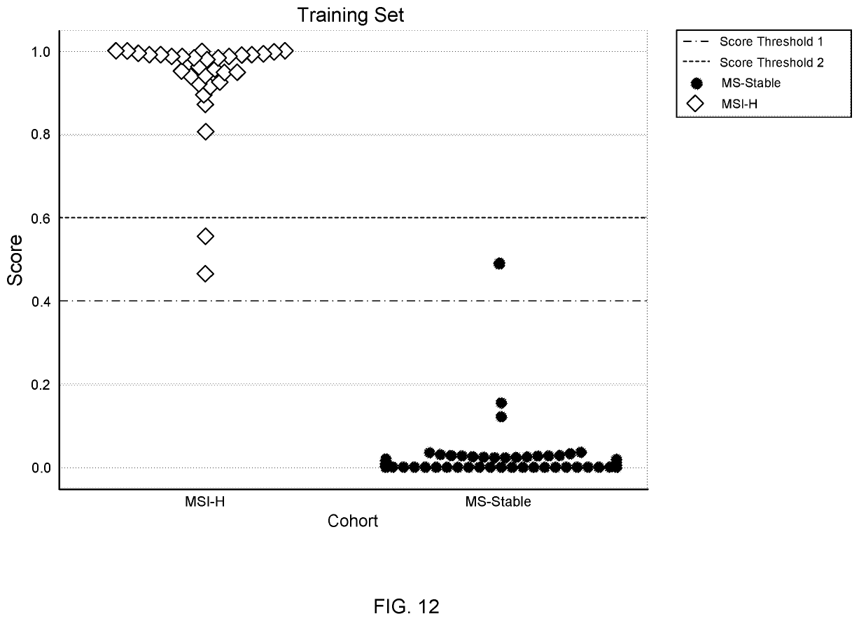

FIG. 12 is a graph plotting experimental results based on the above-described method where test data using known MSI-H and MS-Stable were used to train the random forest classifier as previously described. The results shown are from the predictions obtained from the final cross-validation ensemble random forest (RF) classifier for samples included in training. The samples known to be MSI-H are plotted on the left side of the graph whereas the samples known to be MS-Stable (circles) are plotted on the right side of the graph. With this distribution, at least one threshold for distinguishing scores considered MSI-H and MS-Stable is determined at step 110.

In step 110 an optimum threshold for separating the 2 classes (MSI-H vs MS-Stable groupings) can be automatically determined by maximizing the true positive rate and minimizing the false positive rate (i.e. furthest point from the diagonal on a receiver operating curve). The optimum threshold value for the above model was determined to be 0.5 (rounded to the first decimal point) with the presently used training set of data. However, from FIG. 12, it can be seen that there are 3 samples from both groups with scores close to the 0.5 optimum threshold. If a 0.1 adjustment value around the 0.5 threshold is applied, 2 score thresholds as shown in FIG. 12 (i.e., score threshold 1 can be 0.4 while score threshold 2 can be 0.6) can be determined based on the distribution of scores across all MSI-H and MS-stable training samples to separate out samples with probability scores close to the 0.5 threshold from samples with high confidence probability scores (i.e., probability scores closer to 0 or 1).

Therefore in the presently shown example, sample scores greater than the threshold 2 value of 0.6 have a predicted MSI status=MSI-H, sample scores less than the threshold 1 value of 0.4 have a predicted MSI status=MS-Stable, and sample scores between the 2 thresholds have a predicted MSI status=possible evidence of MSI.

The above mentioned adjustment values can be executed automatically by a configured algorithm once the receiver operating curve has been determined and the probability scores are analyzed, with particular focus on the scores proximate the optimum threshold value and the spread of these scores above and below the optimum threshold. In the example above for FIG. 12, the difference of the 3 probability scores in question relative to the optimum threshold are determined by the system, and the greatest difference above and below the optimum threshold rounded up/down to the first decimal point are selected as the pair of score thresholds.

In an alternate example, if the two MSI-H scores between 0.6 and 0.4 of FIG. 12 are shifted just above 0.6 and below 0.4 respectively, then the adjustment value can be determined by the algorithm to be 0.2 above and below the optimum threshold of 0.5 when rounding up/down the probability scores to the first decimal point.

In a further alternate example, if just the MSI-H score of FIG. 12 is slightly above 0.6, then one score threshold is determined by the algorithm to be 0.2 over the optimum threshold and the other score threshold is determined to be 0.1 below the optimum threshold.

In another alternate example, consider the MS-Stable score between 0.6 and 0.4 in FIG. 12 was not present as part of the training data. In such a hypothetical example the optimum threshold is determined to be about 0.4, and there is no more MSI-possible region.

After the suitable thresholds have been determined, the training phase is deemed completed. Now the detection of MSI in real samples can be initiated, which is a function of the detection phase of the MSI detection system of the present embodiments. Hence once the random forest classifier has been trained in the training phase, it can be used to process new data from an actual sample to determine if the sample is at least MSI-H or MS-Stable in the detection phase.

It is assumed that the real sample and the negative control sample have both been subjected to PCR and sequencing in the same run. In the presently described embodiments, reference to having samples subjected to the same sequencing run includes both PCR and sequencing processes.

The MSI detection phase starts with a pre-processing step that includes filtering of poor-quality reads, followed by an alignment of good quality reads to a reference genome. Following these steps is execution of the trained machine learning classifier that returns the probability that a sample is MSI-H (as opposed to the rest i.e., MSS/MSI-L, collectively referred to as MS-Stable) based on the distribution of repeats in 21 MS loci in the 20 MS amplicons. All of these steps require the use of a computing system.

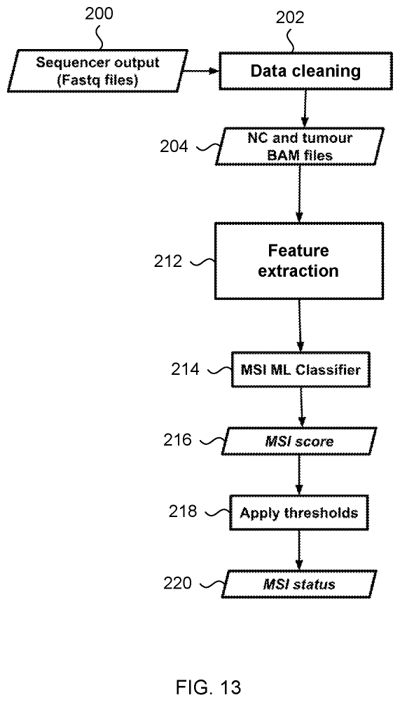

The detection phase method according to the present embodiment is shown in the flowchart of FIG. 13. The following description refers to the different steps of the method shown in FIG. 13. The computing system requires a path to the directory where the tumor samples fastq files and the negative control (NC) sample fastq files are stored. The detection phase can be broken down into the following sub-phases of 1. Preprocessing and 2. MSI ML classifier execution; which are explained in further detail below.

### 1. Preprocessing

The preprocessing sub-phase begins with the input of sequencer outputs 200, which are then data cleaned at step 202. This data cleaning step 202 includes filtering out poor-quality/off-target reads and then aligning to the reference genome GRCh37/HG19 using an alignment software tool to generate a standard file 104 for each sample containing sequence data. For example, this can be performed using samtools which will generate a BAM file. After data cleaning 202, a BAM file for each sample is generated at step 204, and will subsequently be used as input for the MSI caller. The MSI caller is the resulting trained classifier, which in the present examples is a random forest classifier.

The described steps of the preprocessing phase can be executed anywhere and anytime, with any algorithms for filtering and aligning being used. The above-described preprocessing is just one example set of steps that can be executed. Alternate techniques can be used as would be known to those of skill in the bioinformatics/genomics field.

### 2. MSI ML Classifier

The MSI ML classifier execution phase is next. Using the BAM files, or any other type of file, as input, the method proceeds to a feature extraction step 212, where the distribution of repeats are calculated for the samples and for the NC, and then the distances relative to NC are calculated for each MSI loci using the BAM file with MANTIS v1.0.5 which are then used as features for the MSI ML classifier. It is assumed for the present embodiments that the classifier being used is the random forest classifier which has been previously trained. At step 214 the final MSI prediction is calculated by taking the average of the predictions from the 10 models obtained from cross-validations of the random forest classifier (i.e., cross-validation ensemble). The MSI ML classifier produces as output a probability of the sample being MSI-H, which is the MSI score 216 of the sample which is recorded. Reportable thresholds, such as the ones determined from the training shown in FIG. 12, are applied to the MSI score at step 218 to determine the MSI status of the sample. Example thresholds appear in Table 1 below for the FIND IT assay, and shown in FIG. 12.

TABLE 1

| MSI Score Range | FIND IT MSI Status |
|---|---|
| score > 0.6 | MSI-H |
| 0.4 < score <= 0.6 | Possible evidence of MSI |
| score <= 0.4 | MS-Stable |

The output from the MSI ML classifier is recorded in a file 220 with a structured format, such as into columns. The columns in this file are (1) Sample, (2) Score (ML Classifier), and (3) MSI status.

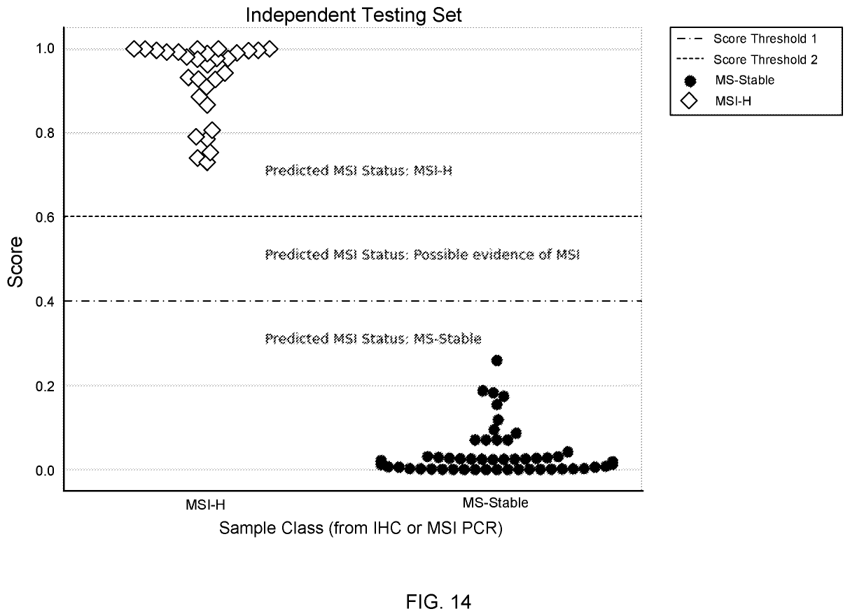

FIG. 14 is a graph plotting experimental results based on the above-described method where an independent testing set of data not included in the training set were used. Similar to the training data which included known MSI-H and MS-Stable samples, this independent testing set of data includes known MSI-H and MS-Stable samples. For convenience, the graph shows samples from the MSI-H cohort positioned to the left side of the graph, whereas the samples from the MS-Stable cohort have been positioned to the right side of the graph. The y-axis shows the prediction scores obtained from the MSI ML classifier. It is recalled that the two thresholds of 0.4 and 0.6 were determined from the training set of data. From the graph, there is a clear separation of the samples from the MSI-H cohort having prediction scores above 0.6 and the samples from the MSI-Stable cohort having prediction scores below 0.4.

It can be concluded from the results shown in FIG. 14 that the training of the random forest classifier using the known MSI-H and MS-Stable samples is correct because the independent testing set of data has been classified as expected.

In a real-world situation where a sample is classified as MSI-H, the result “MSI-H” can be output clearly in the output file or onto a display, with information that the finding is indicative of the tested sample being sensitive to immune modulation-checkpoint inhibitor treatment. More specifically, the indication can include information that cancer treatment options are available for the MSI-H tumor based on the test for this genomic marker.

On the other hand, if the sample is classified as MS-Stable, the output file or the display will simply be “MS-Stable” with no treatment suggestions being given based on the test for this genomic marker. Other treatment recommendations may be given for these samples based on tests for other genomic markers in the assay.

From the experiments conducted, it has been found that the MSI detection system of the present embodiments can predict the MSI status with >99% accuracy when analyzing 21 MS loci. By comparison, other known systems require analysis of at least 100 loci. This is because the system of the present embodiments utilizes distributions of repeats as features in a machine learning model, and in particular a random forest classifier machine learning model. Additionally, normalization of the features is achieved using the negative control in every run. Another advantage of the method is that a training set of MSI status known (from IHC or MSI PCR) samples sequenced with the FIND IT v5 assay are used to establish a baseline in order to identify MSI-H samples without requiring a second matched normal sample from the same patient (healthy tissue from the same individual as the sample under test).

There are many advantages of the presently described MSI detection system over known methods for classifying MSI-H, MS-Stable and possible MSI samples.

As mentioned earlier, the present system requires only one sample from the patient, that being a tumour sample, while most other methods require two samples from the same patient (i.e., tumour and normal tissue/cells), which doubles the sequencing cost.

The present system uses a small number of MS sites, such as 21 by example. In a targeted panel, a sample will be allocated roughly a fixed number of sequencing reads (sequencing bandwidth). Therefore, it is desirable to detect MSI using fewer MS sites to allow for more reads to be allocated to regions where higher numbers of reads are required (e.g., for detecting low prevalent single nucleotide variants). Having to use >100 MS sites for MSI detection with existing methods, which is required to gain comparable performance to the methods of the present embodiments, is a disadvantage because more reads will need to be allocated to MS detection and the number of reads allocated to other regions will be reduced.

As mentioned above, experiments were conducted with the previously described MSI detection system embodiments using an analysis of 21 MS loci. In alternative embodiments, an analysis of greater than 21 MS loci can be used instead with at least the same or better accuracy being obtained. At less than 21 MS loci, it is predictable that the level of performance may decrease. Therefore, where higher sensitivity/LOD (limit of detection) is not required, fewer than 21 MS loci can be used.

With that said, the present system does not need to select the most predictive loci: Some MS sites are more predictive of microsatellite instability in a sample than others (e.g., repeat sequences >10 bp in length, mononucleotide repeat sequences, etc.). Due to limitations in the primer/amplicon design process for targeted assays, it may not be feasible to choose the most predictive MS sites (e.g., if they are in GC rich regions, if they affect performance of other amplicons in the assay, if they form primer-dimers, etc.). Systems such as MANTIS and MSIsensor give equal weights to all amplicons and thus need to filter for more predictive sites by imposing thresholds (e.g., minimum repeat length=10, use only homopolymers). However, because the presently described ML method learns to give different feature importance (weights) to the different MS sites, a mix of more predictive and less predictive loci does not impact the accuracy of detecting MSI-H samples.

The normalization of features using the negative control reduces noise at noisy MS sites. Due to limitations in primer design, the MS site may be in a noisy region (e.g., regions close to the ends of the reads have more sequencing errors). However, these errors are also present in the negative control, so when the features are normalized by the negative control the effect of these errors are canceled out.

The normalization of features using the negative control further eliminates run-to-run variation. Error profiles in sequencing runs may vary depending on the library preparation process of a particular lab and the sequencer version used (e.g., Illumina NextSeq vs MiSeq). One of the disadvantages of some of the existing ML based and non-ML based systems that establish a baseline for MSI detection without requiring a tumour and matched normal sample from the same patient is that a new training set/dataset for establishing baseline will need to be generated before that system can be used in a different lab or on a different sequencer version (e.g. mSINGS). However, with the presently described system the features are normalized using the negative control that is included in every sequencing run of the FIND IT assay. The negative control goes through the same library preparation process and is run on the same sequencer as the sample of interest on the same sequencing run, which allows for the same training set and ML model to be used when the assay is sequenced at a different lab or on a different sequencing instrument.

### MSI Analytical and Clinical Validation Results

The previously described MSI detection system has been executed on Formalin-Fixed Paraffin-Embedded (FFPE) samples, but is not necessarily limited to being executed on FFPE samples. Testing has shown that the above-described system can be executed successfully on cfDNA in plasma samples.

To better exemplify utility of the MSI detection system, with the previously described method for detecting microsatellite instability high (MSI-H) tumours, detailed results of an analytical and clinical validation study are included in this section. Analytical validation is performed using commercial reference standard samples (samples that are specifically developed for the purpose of validating novel MSI detection systems and assays) and well characterized FFPE treated cell-lines. Clinical validation is performed using clinical FFPE tumor samples from cancer patients.

For the analytical validation the following performance metrics are calculated:
Accuracy, NPV and PPV (standard definitions apply)
Sensitivity—Percentage of true positive (MSI-H) samples that are detected as positive.
Specificity—Percentage of true negative samples that are detected as negative.
Repeatability—Results from repeats of the same samples sequenced on the same sequencing run (referred to as intra-run repeats) are tested for concordance.
Reproducibility—Results from repeats of the same samples sequenced on different sequencing runs, on different days, prepared by different technologists, and on different sequencers (referred to as inter-run repeats) are tested for concordance.
Limit of Detection (LOD)—The minimum tumor fraction required to detect MSI-H status in a sample.
Results at lower inputs—The sensitivity/specificity/accuracy values at inputs lower (25 ng-4 ng) than the standard input (i.e., 25 ng).

For the clinical validation all the above metrics are calculated except for LOD and results at lower inputs due to limitations in sample availability for the required experimental design.

Details of the validation design, the samples used, and the results for each of the performance metrics are described below for the MiSeq sequencer.

### Validation on the Illumina MiSeq Sequencer

The validation runs that were sequenced for this study were performed by multiple technologists, on different days, and two Illumina MiSeq instruments. All experiments were performed in the Canexia Health CAP, CLIA, DAP certified laboratory using locked down Find It assay standard operating procedures.

### Analytical Validation

MSI-H and MS-Stable commercial reference standards and MSI status known FFPE cell lines were used for the analytical validation of the MSI detection system embodiments (Table 2). A total of 37 (11 unique) samples were sequenced and used for calculating sensitivity and specificity. Six samples were run as inter-run repeats and intra-run repeats, which were used for calculating reproducibility and repeatability.

Depending on the score value, which is the probability of a sample being MSI-H, samples can be classified into two positive classes: MSI-H (score>0.6) or possible evidence of MSI (0.4<score<=0.6). The distinction between these two classes is that orthogonal validation is required for samples in the possible evidence of MSI class and it is not required for the MSI-H class. All performance metrics are calculated for when only the MSI-H class is considered as positive and for when both the MSI-H class and the possible evidence of MSI class are considered as positive.

TABLE 2
MSI samples used in the analytical validation

| | Number | |
|---|---|---|
| Sample Type | of samples | Description |
| Commercial | 2 | Horizon MSI FFPE DNA Reference |
| Reference | | Standard (HD830) |
| Standards | | Horizon MSS FFPE DNA Reference |
| | | Standard (HD831) |
| Cell lines | 9 | SW48 FFPE cell line pellet (MSI) |
| | | SNU-C2B FFPE cell line pellet (MSI) |
| | | HCT15 FFPE cell line pellet (MSI) |
| | | HCT116 FFPE cell line pellet (MSI) |
| | | LS174T FFPE cell line pellet (MSI) |
| | | SW403 FFPE cell line pellet (MSS) |
| | | Colo201 FFPE cell line pellet (MSS) |
| | | Coriell 1 ‘Genome in a Bottle’ |
| | | NA12878/HG001 (MSS) |
| | | Coriell 2 ‘Genome in a Bottle’ |
| | | NA24385/HG002 (MSS) |

### Analytical Accuracy

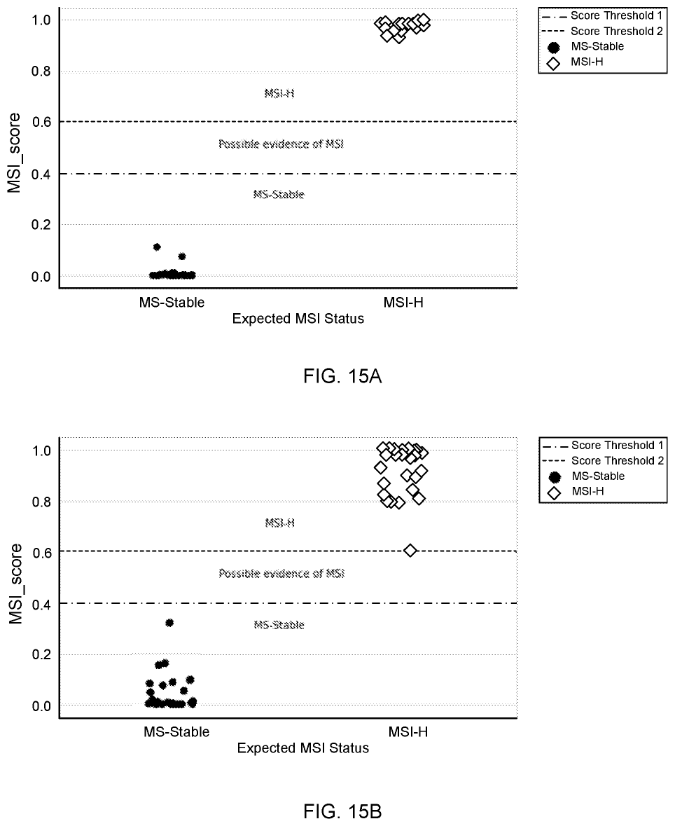

The accuracy of the MSI detection system embodiments was determined for when only the MSI-H class is considered as positive and for when both the MSI-H class and the possible evidence of MSI class are considered as positive (Table 3). For both positive classes, the PPV, NPV and accuracy are 100%. A summary of the MSI analytical validation results is shown in FIG. 15A. The previously used symbol convention for MSI-H samples shown as diamonds with MS-Stable samples shown as circles are used here.

TABLE 3
MSI analytical accuracy

| | # of | | | | | | | Accu- |
|---|---|---|---|---|---|---|---|---|
| Positive Class | Samples | TP | TN | FP | FN | PPV | NPV | racy |
| MSI-H only | 37 | 16 | 21 | 0 | 0 | 100% | 100% | 100% |
| MSI-H and Possible | 37 | 16 | 21 | 0 | 0 | 100% | 100% | 100% |
| evidence of MSI | | | | | | | | |

### Analytical Repeatability

MSI analytical repeatability was calculated using intra-run repeats of 6 different samples with the same input (25 ng per primer pool) and the output results for all replicates were concordant (Table 4).

TABLE 4
MSI analytical repeatability of the assay

| | # of | | | Repeatability |
|---|---|---|---|---|
| Positive Class | samples | Concordant | Discordant | (sample level) |
| MSI-H | 6 | 6 | 0 | 100% |
| MSI-H and Possible | 6 | 6 | 0 | 100% |
| evidence of MSI | | | | |

### Analytical Reproducibility

MSI analytical repeatability was calculated using inter-run repeats of 6 different samples with the same input (25 ng per primer pool) and the output results for all replicates were concordant (Table 5).

TABLE 5
MSI analytical reproducibility of the assay

| | # of | | | Reproducibility |
|---|---|---|---|---|
| Positive Class | Samples | Concordant | Discordant | (sample level) |
| MSI-H | 6 | 6 | 0 | 100% |
| MSI-H and Possible | 6 | 6 | 0 | 100% |
| evidence of MSI | | | | |

### Analytical Sensitivity

MSI analytical sensitivity was calculated using 16 MSI-H samples and all samples were detected as MSI-H, therefore the sensitivity is 100% (Table 6).

TABLE 6
MSI analytical sensitivity of the assay

| | # of | | | |
|---|---|---|---|---|
| Positive Class | Samples | TP | FN | Sensitivity |
| MSI-H only | 16 | 16 | 0 | 100% |
| MSI-H and Possible evidence of MSI | 16 | 16 | 0 | 100% |

### Limit of Detection (LOD)

The LOD for MSI was determined in terms of tumour fraction. 3 MSI-H FFPE cell lines were diluted with an MS-Stable FFPE cell line to create samples with 10%, 15% and 20% tumour fraction. When considering only the MSI-H class as positive, and when considering both the MSI-H class and the possible evidence of MSI class as positive, the LOD was determined to be at 15% tumour fraction (Table 7 and 8).

TABLE 7
MSI LOD (considering only the MSI-H class)
MSI-H only

| Tumour | Expected # of | Observed # of | |
|---|---|---|---|
| Fraction | MSI-H samples | MSI-H samples | Sensitivity |
| 20% | 3 | 3 | 100% |
| 15% | 3 | 3 | 100% |
| 10% | 3 | 0 | 0% |

TABLE 8
MSI LOD (considering the MSI-H class and
the possible evidence of MSI class)
MSI-H and Possible evidence of MSI

| Tumour | Expected # of | Observed # of | |
|---|---|---|---|
| Fraction | MSI-H samples | MSI-H samples | Sensitivity |
| 20% | 3 | 3 | 100% |
| 15% | 3 | 3 | 100% |
| 10% | 3 | 2 | 66.66% |

### Analytical Specificity

MSI analytical specificity was calculated using 21 MS-Stable samples and all samples were detected as MS-Stable (Table 9).

TABLE 9
MSI analytical specificity

| | # of | | | |
|---|---|---|---|---|
| Positive Class | samples | TN | FP | Specificity |
| MSI-H only | 21 | 21 | 0 | 100% |
| MSI-H and Possible evidence of MSI | 21 | 21 | 0 | 100% |

### Summary of Results at Different Inputs

The MSI/MSS Horizon reference standard samples, an MSI cell line (LS174T) and an MSS cell line (SW403) were run with inputs of 25 ng, 15 ng, 10 ng, and 4 ng and 2 Coriell ‘Genome in a bottle’ samples were run with 25 ng and 10 ng inputs. Additional, 3 MSI cell lines (SNU-C2B, HCT15, HCT116) and an MSS cell line (Colo201) were sequenced at 25 ng input. The expected MSI status was detected in all samples (Table 10).

TABLE 10
MSI table

| | | | # of | | | | |
|---|---|---|---|---|---|---|---|
| | Input | | Samples | Sensitivity | Specificity | PPV | NPV |
| MSI | 25 | ng | 23 | 100% | 100% | 100% | 100% |
| | 15 | ng | 4 | 100% | 100% | 100% | 100% |
| | 10 | ng | 6 | 100% | 100% | 100% | 100% |
| | 4 | ng | 4 | 100% | 100% | 100% | 100% |

### Clinical Validation

### Validation Design

Clinical validation of the MSI component was performed using 58 (44 unique) orthogonally validated FFPE clinical endometrial and colorectal cancer samples (Table 11). 4 samples were sequenced 4 as inter-run repeats and intra-run repeats, which were used for calculating reproducibility and repeatability of the assay. 8 samples were sequenced with lower input (10 ng-24 ng) and the rest were sequenced with 25 ng input.

As with the analytical validation, all performance metrics for the clinical validation are calculated for when only the MSI-H class is considered as positive and for when both the MSI-H class and the possible evidence of MSI class are considered as positive.

TABLE 11
MSI samples used in the clinical validation

| Tumor | Number of clinical samples | Intra-run | Inter-run |
|---|---|---|---|
| Diagnosis/type | tested | repeats | repeats |
| Colorectal | 38 (17 MSI, 21 MSS) | 2 | 2 |
| Endometrial | 6 (2 MSI, 4 MSS) | 2 | 2 |

### MSI Orthogonal Validation Method:

Samples used for MSI analysis were orthogonally validated using immunohistochemistry (expression of mismatch repair genes, i.e., MMRnormal vs MMRd) on tumour tissue and/or Promega MSI PCR using matched tumour/normal (MSS vs MSI-H).

### Clinical Accuracy

The accuracy of the MSI detection system embodiments was determined for when only the MSI-H class is considered as positive and for when both the MSI-H class and the possible evidence of MSI class are considered as positive (Table 12). For both positive classes the PPV, NPV and accuracy were 100%. A summary of the MSI clinical validation results is shown in FIG. 15B. The previously used symbol convention for MSI-H samples shown as diamonds with MS-Stable samples shown as circles are used here.

TABLE 12
MSI clinical accuracy

| | # of | | | | | | | Accu- |
|---|---|---|---|---|---|---|---|---|
| Positive Class | samples | TP | TN | FP | FN | PPV | NPV | racy |
| MSI-H only | 58 | 27 | 31 | 0 | 10 | 100% | 100% | 100% |
| MSI-H and Possible | 58 | 27 | 31 | 0 | 0 | 100% | 100% | 100% |
| evidence of MSI | | | | | | | | |

### Clinical Repeatability

MSI clinical repeatability was calculated using intra-run repeats of 4 sample with the same input (25 ng) and the results for all replicates were concordant, therefore the repeatability is 100% (Table 13).

TABLE 13
MSI clinical repeatability

| | # of | | | Repeatability |
|---|---|---|---|---|
| Positive Class | samples | Concordant | Discordant | (sample level) |
| MSI-H only | 4 | 4 | 0 | 100% |
| MSI-H and Possible | 4 | 4 | 0 | 100% |
| evidence of MSI | | | | |

### Clinical Reproducibility

MSI clinical reproducibility was calculated using inter-run repeats of 4 different samples with the same input (25 ng) and the results for all replicates were concordant, therefore the repeatability is 100% (Table 14).

TABLE 14
MSI clinical reproducibility

| | # of | | | Reproducibility |
|---|---|---|---|---|
| Positive Class | samples | Concordant | Discordant | (sample level) |
| MSI-H only | 4 | 4 | 0 | 100% |
| MSI-H and Possible | 4 | 4 | 0 | 100% |
| evidence of MSI | | | | |

### Clinical Sensitivity

MSI clinical sensitivity was calculated using 27 (19 unique) MSI-H samples (10-25 ng per primer pool input) and all samples were detected as MSI-H. The clinical sensitivity is 100% (Table 15).

TABLE 15
MSI clinical sensitivity of the assay

| | # of | | | |
|---|---|---|---|---|
| Positive Class | samples | TP | FN | Sensitivity |
| MSI-H only | 27 | 27 | 0 | 100% |
| MSI-H and Possible evidence of MSI | 27 | 27 | 0 | 100% |

### Clinical Specificity

MSI clinical specificity was calculated using 31 (26 unique) MS-Stable samples (10-25 ng per primer pool input) and they were all classified as MS-Stable. The clinical specificity is 100% (Table 16).

TABLE 16
MSI clinical specificity

| | # of | | | |
|---|---|---|---|---|
| Positive Class | samples | TN | FP | Specificity |
| MSI-H only | 31 | 31 | 0 | 100% |
| MSI-H and Possible evidence of MSI | 31 | 31 | 0 | 100% |

### CONCLUSION

MSI status is reported based on a probability score (i.e., probability of the sample being MSI-H) for samples that meet the minimum coverage requirement for microsatellite amplicons. Samples that do not meet the coverage criterion are not reportable for MSI. The reportable MSI statuses are: “MSI-H” (score>0.6); “Possible evidence of MSI” (0.4<score<=0.6); and “MS-Stable” (score<=0.4). When the MSI-H class is considered as the positive class and when both the MSI-H class and the possible evidence of MSI class are considered as positive, MSI-H samples (10 ng-25 ng input per pool) can be detected with high sensitivity, specificity, accuracy, NPV, PPV, repeatability, and reproducibility for samples with >15% tumour fraction. In conclusion, these results indicate the MSI detection system can accurately identify patients with MSI-H tumours. When used in a clinical setting, these patients can then be directed to treatments such as immune-checkpoint inhibitors which have been shown to have significantly higher success rates compared to other treatments for this tumour subtype.

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

The term “plurality” as used herein means more than one, for example, two or more, three or more, four or more, and the like.

The use of the word “a” or “an” when used herein in conjunction with the term “comprising” may mean “one”, but it is also consistent with the meaning of “one or more”, “at least one”, and “one or more than one”.

As used herein, the terms “comprising”, “having”, “including”, and “containing”, and grammatical variations thereof, are inclusive or open-ended and do not exclude additional, unrecited elements and/or method steps. The term “consisting essentially of” when used herein in connection with an apparatus, system, composition, use or method, denotes that additional elements and/or method steps may be present, but that these additions do not materially affect the manner in which the recited apparatus, system composition, method or use functions. The term “consisting of” when used herein in connection with an apparatus, system, composition, use or method, excludes the presence of additional elements and/or method steps. An apparatus, system composition, use or method described herein as comprising certain elements and/or steps may also, in certain embodiments consist essentially of those elements and/or steps, and in other embodiments consist of those elements and/or steps, whether or not these embodiments are specifically referred to.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be affected to the particular embodiments by those of skill in the art. The scope of the claims should not be limited by the particular embodiments set forth herein, but should be construed in a manner consistent with the specification as a whole.

1. A method for classifying a tissue sample of a person as being microsatellite instability high (MSI-H) without using normal tissue from the same person, comprising:
training a machine learning classifier algorithm using known MSI-H samples and known MS-Stable samples to learn a baseline distribution of repeats in microsatellite regions of genomes relative to a first negative control sample at multiple corresponding MS loci, where each of the known MSI-H samples and the known MS-Stable samples are scored with a probability of being MSI-H and the first negative control sample is known to be MS-Stable;
setting a threshold probability score in response to groupings of probability scores of the known MSI-H samples and the known MS-Stable samples where the probability scores greater than the threshold probability score are classified as MSI-H;
determining a distance of distributions of repeats in the tissue sample normalized to a second negative control sample at multiple MS loci, where both the tissue sample and the second negative control sample are part of the same sequencing run and the second negative control sample is known to be MS-Stable;
executing the trained machine learning classifier algorithm on the distance distributions for the tissue sample to provide a corresponding probability score;
determining the tissue sample as MSI-H if the score is greater than the threshold probability score; and
outputting the MSI-H status of the sample as being indicative of sensitive to immune modulation-checkpoint inhibitor treatment if the score is greater than the threshold.

2. The method of claim 1, wherein training the machine learning classifier algorithm
includes preparing training data for learning by the machine learning classifier algorithm by executing a sequencing run on a plurality of the known MSI-H samples, a plurality of the known MS-Stable samples, and the first negative control sample in the same run.

3. The method of claim 1, wherein training the machine learning classifier algorithm
includes preparing training data for learning by the machine learning classifier algorithm by executing multiple sequencing runs, each run including a plurality of the known MSI-H samples, a plurality of the known MS-Stable samples, and the first negative control sample.

4. The method of claim 1, wherein training the machine learning classifier algorithm includes preparing training data for learning by the machine learning classifier algorithm by
executing multiple sequencing runs, each run including a plurality of the known MSI-H samples and the first negative control sample, or a plurality of the known MS-Stable samples and the first negative control sample.

5. The method of claim 4, wherein preparing the training data includes
determining distances between distribution of repeats at the multiple MS loci in each of the known MSI-H samples relative to the first negative control sample, and
determining distances between distribution of repeats at the same multiple MS loci in each of the known MS-Stable samples relative to the first negative control sample.

6. The method of claim 5, wherein the distances are determined using a distance metric.

7. The method of claim 6, wherein the distance metric is a stepwise difference distance metric expressed by
$$d = {\sum }_{γϵ ( {R}_{T} ⋃ {R}_{NC} )} \vert {T}_{γ} - {NC}_{γ} \vert$$
where
Tγ is the fraction of reads with repeats of length γ in the tumour sample at a given MS locus
NCγ is the fraction of reads with repeats of length γ in the NC sample at the same MS locus

8. The method of claim 5, wherein the threshold probability score is determined by obtaining the optimum threshold that maximizes the true positive rate and minimizes the false positive rate and selecting an adjustment value above it where scores greater than the threshold probability score are classified as MSI-H.

9. The method of claim 8, wherein the threshold probability score is a first threshold, and a second threshold is determined by the adjustment value lower than the optimum threshold and samples with scores below this second threshold are classified as MS-Stable.

10. The method of claim 9, wherein the adjustment value is determined by a spread of training data scores proximate the optimum threshold.

11. The method of claim 10, wherein the adjustment value is a first adjustment value over the optimum threshold and a second adjustment value is determined as below the optimum threshold.

12. The method of claim 11, wherein the first threshold and the second threshold are different, the first threshold is greater than the second threshold, and scores falling between the first threshold and the second threshold are classified as possible MSI.

13. The method of claim 12, wherein outputting the MSI-H status includes outputting the MS-Stable status of the person as MS-Stable or outputting the possible evidence of MSI status of the person as requiring orthogonal testing.

14. The method of claim 12, wherein training the machine learning classifier algorithm includes providing a subset of the training data for execution by the machine learning classifier algorithm.

15. The method of claim 14, wherein training further includes validating the trained machine learning classifier algorithm by providing a remainder of the training data that is not the subset of the training data and comparing the probability scores against expected classification results.

16. The method of claim 2, wherein the machine learning classifier algorithm is a random forest classifier.

17. The method of claim 16, wherein distributions of the repeats for less than 100 MS loci are used for training the machine learning classifier algorithm.

18. The method of claim 17, wherein distributions of the repeats for 21 MS loci are used for training the machine learning classifier algorithm.

19. A method of training a machine learning classifier algorithm to classify a tissue sample from a person as being at least MSI-H and indicative of being sensitive to immune modulation-checkpoint inhibitor treatment, comprising:
executing multiple sequencing runs, each run including
a plurality of the known MSI-H samples and a negative control sample, where the negative control sample is known to be MS-Stable,
a plurality of the known MS-Stable samples and the negative control sample, or
a combination of the known MSI-H samples the known MS-Stable samples and the negative control sample;
determining distances between distribution of repeats at multiple MS loci in each of the known MSI-H samples relative to the negative control sample;
determining distances between distribution of repeats at the same multiple MS loci in each of the known MS-Stable samples relative to the negative control sample; and
providing the distances and their average as features for the machine learning classifier algorithm to learn a baseline distribution of the repeats in the multiple MS loci from the known MSI-H samples and the known MS-Stable samples, and scoring each sample with a probability of being MSI-H.

20. The method of claim 19, wherein the machine learning classifier algorithm is a random forest classifier using the distribution of the repeats as features.

21. The method of claim 20, wherein distributions of the repeats for less than 100 MS loci are used for training the machine learning classifier algorithm.

22. The method of claim 21, wherein distributions of the repeats for 21 MS loci are used for training the machine learning classifier algorithm.

23. A method for classifying a tissue sample of a person as being microsatellite instability high (MSI-H) without using normal tissue from the same person, using a random forest machine learning classifier algorithm trained using known MSI-H samples and known MS-Stable samples to learn a baseline distribution of repeats in microsatellite regions of genomes relative to a first negative control sample known to be MS-Stable at multiple corresponding MS loci, comprising:
determining a distance of distributions of repeats in the tissue sample normalized to a second negative control sample at multiple MS loci, where both the tissue sample and the second negative control sample are part of the same sequencing run and the second negative control sample is known to be MS-Stable;
executing the trained machine learning classifier algorithm on the distance distributions for the tissue sample to provide a corresponding probability score;
comparing the probability score of the tissue sample to a first predetermined threshold probability score and a second predetermined threshold probability score; and
outputting the MSI-H status of the person as a finding indicative of sensitivity to immune modulation-checkpoint inhibitor treatment if the probability score is greater than the first predetermined threshold, or outputting the status of the person as a clinically relevant status if the probability score is less than the second predetermined threshold.

24. The method of claim 23, wherein the machine learning classifier algorithm is trained and executes using distributions of the repeats for a set number of MS loci less than 100.

25. The method of claim 23, wherein the first predetermined threshold is greater than the second predetermined threshold, and outputting further includes outputting a possible evidence of MSI status of the person as requiring orthogonal testing when the probability score of the tissue sample is at or between the first predetermined threshold and the second predetermined threshold.
